(19) United States
(12) Patent Application Publication
(10) Pub. No.: US 2005/0100588 A1
(43) Pub. Date: May 12, 2005

(54) Self-adhesive matrix plaster containing an active ingredient and based on polyurethane gels

(75) Inventors: Holger Kartheus, Hamburg (DE); Michael Schink, Hamburg (DE); Jurgen-Christian Quandt, Nordende (DE); Peter Philipp, Oststeinbek (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/735,310

(22) Filed: Dec. 12, 2003

Related U.S. Application Data
(63) Continuation of application No. PCT/EP02/06429, filed on Jun. 12, 2002.

(30) Foreign Application Priority Data
Jun. 13, 2001 (DE) 10128685.6

(51) Int. Cl. A61K 9/70; A61F 13/00

(52) U.S. Cl. 424/449

(57) ABSTRACT

The present invention is a self-adhesive matrix patch for controlled delivery of active ingredients to the skin. The self-adhesive matrix patches comprises a polyurethane gel matrix, wherein the polyurethane gel matrix comprises at least one active ingredient and at least one penetration enhancer.

The invention relates to self-adhesive, active ingredient-containing, matrix patches based on polyurethane gels, in particular with circulation-promoting active ingredients.

Active ingredient-containing patches for transdermal administration have been described many times in the literature and in patents.

Transdermal patch systems can be differentiated for example according to their construction.

In the membrane-controlled transdermal therapeutic systems, a separate active ingredient reservoir is located between an outer impermeable cover layer and a semipermeable control membrane which controls the release of the active ingredient into the skin and is combined with an additional adhesive layer for fixation to the skin.

Since the individual components of these systems with a complicated construction must be carefully matched with one another, manufacture is costly.

In the matrix-controlled systems, an intrinsic active ingredient reservoir is constructed by homogeneous dispersion of the active ingredient in a polymer matrix or a gel matrix. In this case, the polymer matrix or gel matrix ideally has self-adhesive properties so that it is unnecessary to fix the matrix on the skin by additional application of an adhesive layer. In the simplest case, the active ingredient-containing matrix is located between a cover layer firmly anchored thereto and a detachable separating layer.

The active ingredient is normally blended homogeneously into the polymer matrix or gel matrix by dissolving, dispersing, suspending, extruding, kneading, mixing or similar processes, in some cases at elevated temperature.

The use of polyurethanes for controlled delivery of active ingredient is described only in a few cases (Lamba, Woodhouse, Cooper, “Polyurethanes in Biomedical Applications”, CRC Press, 1998, p. 240).

EP 0 057 839 A1 describes polyurethane gels into which a variety of active ingredients may also be incorporated, and describes their use as active ingredient carriers with a depot effect.

The hydrophilic, self-adhesive polyurethane gel compositions described in WO 97/43328 A1 are used with preference as active-ingredient-free wound contact materials for treating chronic wounds. Examples of their qualities include skin friendliness, adhesion, even over a prolonged period of application, and pain-free removability following application.

EP 0 016 652 A1 describes an active ingredient composition which is prepared by reacting a polyethylene oxide with polyfunctional isocyanates and which constitutes a crystalline hydrogel in the dry form. Swelling a polymeric carrier produced in this way in a solution of an active substance, and then drying it, produces a composition featuring controlled release.

WO 96/31551 A1 concerns itself with polyurethane microgels comprising active substances, proteins for example, said microgels being able to swell in water and, in doing so, release the active ingredient.

WO 91/02763 A1 and WO 94/22934 A1 also concern themselves with compositions for the controlled delivery of active substances from hydrogels based on polyurethane-ureas.

The known polyurethanes comprising active substance are, moreover, products which do not have self-attaching properties.

Circulation-promoting active ingredient patches are used for treating rheumatic complaints, muscle strains, and pain in the region of the locomotor apparatus. Known patch systems which act by heating comprise an adhesive based on rubber, hydrocolloid or hydrogel, into which one or more active ingredients having circulation-promoting properties, such as benzyl nicotinate, capsaicin, and nonivamide, have been incorporated.

In addition to controlled release of active ingredient, active ingredient patch systems are also subject to certain requirements concerning the adhesive matrix, such as skin friendliness, adhesion over a long period of application, and pain-free removability, for example. Self-adhesive, hydrophilic polyurethane gels which are employed in the sector of chronic wound healing are particularly good at meeting the last-mentioned requirements. Although these systems have been described, inter alia, as active ingredient carriers, their active ingredient release when using circulation-promoting active ingredients such as nonivamide, benzyl nicotinate, and capsaicin, for example, is low. One reason for this is that the profile of properties of known polyurethane gel products, especially hydrophilic polyurethane gel products, is tailored to moist wound healing—for example, the ability to absorb fluid from the wound—and not to the delivery of active ingredients into intact skin.

It is an object of the invention to provide an active ingredient matrix patch for the controlled delivery of active ingredients to the skin and/or into the wound, said patch being self-adhesive and economic to produce.

This object is achieved by a matrix patch as set forth in claim 1. The dependent claims embrace advantageous variants of the subject matter of the invention.

The present invention provides self-adhesive, active ingredient-containing matrix patches for controlled delivery of active ingredients to the skin or into the wound, having an absorbent, self-adhesive matrix based on polyurethane gels, where the active ingredient is present in the matrix and where penetration enhancers have been added to the matrix.

It is advantageous to add penetration enhancers to the matrix at up to 30% by weight, in particular from 5 to 15% by weight.

The penetration enhancers include, for example, lipophilic solubilizers/enhancers lipophilic solubilizers/enhancers such as decyl oleate, isopropyl myristate and isopropyl palmitate (IPM and IPP), 2-octyldodecanol and/or other fatty acid esters.

Enhancers used with further preference are fatty acid esters (C₈-C₁₈) with short-chain alcohols or fatty alcohols.

Fatty alcohols is a collective term for the linear, saturated or unsaturated primary alcohols (1-alkanols) having 6 to 22 carbon atoms that are obtainable by reduction of triglycerides, fatty acids and/or fatty acid methyl esters.

Fatty alcohols are neutral, colorless, high-boiling, oily liquids or soft colorless masses which are sparingly soluble or insoluble in water but readily soluble in alcohol and ether. The table below gives physicochemical data for the fatty alcohols.

TABLE
Physicochemical data of the fatty alcohols

| | | | | b.p. |
|---|---|---|---|---|
| | | | m.p. | ° C./ |
| Alcohol | Formula | Mᵣ | ° C. | kPa |
| 1-Hexanol | C₆H₁₄O | 102.18 | −51.6 | 157.2 |
| (caproyl alcohol) | | | | |
| 1-Heptanol | C₇H₁₆O | 116.20 | −30.0 | 177 |
| (enanthyl alcohol) | | | | |
| 1-Octanol | C₈H₁₈O | 130.23 | −16.3 | 194.5 |
| (caprylyl alcohol) | | | | |
| 1-Nonanol | C₉H₂₀O | 144.26 | | 212 |
| (pelargonyl alcohol) | | | | |
| 1-Decanol | C₁₀H₂₂O | 158.28 | −7.0 | 229 |
| (capryl alcohol) | | | | |
| 1-Undecanol | C₁₁H₂₄O | 172.31 | 16.3 | 131/2.0 |
| 10-Undecen-1-ol | C₁₁H₂₂O | 170.30 | −2 | 133/2.1 |
| 1-Dodecanol | C₁₂H₂₆O | 186.34 | 23.8 | 150/2.7 |
| (lauryl alcohol) | | | | |
| 1-Tridecanol | C₁₃H₂₈O | 200.36 | | 155/2.0 |
| 1-Tetradecanol | C₁₄H₃₀O | 214.39 | 38.0 | 167/2.0 |
| (myristyl alcohol) | | | | |
| 1-Pentadecanol | C₁₅H₃₂O | 228.42 | 44.0 | |
| 1-Hexadecanol | C₁₆H₃₄O | 242.45 | 49.3 | 190/2.0 |
| (cetyl alcohol) | | | | |
| 1-Heptadecanol | C₁₇H₃₆O | 256.47 | 54.0 | 308 |
| 1-Octadecanol | C₁₈H₃₈O | 270.50 | 59.0 | 210/2.0 |
| (stearyl alcohol) | | | | |
| 9-cis-Octadecen-1-ol | C₁₈H₃₆O | 268.48 | −7.5 | 209/2.0 |
| (oleyl alcohol) | | | | |
| 9-trans-Octadecen-1-ol | C₁₈H₃₆O | 268.48 | 36.5 | 216/2.4 |
| (erucyl alcohol) | | | | |
| 9-cis-Octadecen-1,12-diol | C₁₈H₃₆O₂ | 284.48 | | 182/0.07 |
| (ricinoleyl alcohol) | | | | |
| all-cis-9,12-Octadecadien- | C₁₈H₃₄O | 266.47 | −5 | 153/0.4 |
| 1-ol (linoleyl alcohol) | | | | |
| all-cis-9,12,15-Octadecatrien- | C₁₈H₃₂O | 264.45 | | 133/0.3 |
| 1-ol (linolenyl alcohol) | | | | |
| 1-Nonadecanol | C₁₉H₄₀O | 284.53 | 62 | 167/0.04 |
| 1-Eicosanol | C₂₀H₄₂O | 298.55 | 65.5 | 220/0.4 |
| (arachidyl alcohol) | | | | |
| 9-cis-Eicosen-1-ol | C₂₀H₄₀O | 296.54 | | 209/2.0 |
| (gadoleyl alcohol) | | | | |
| 5,8,11,14-Eicosatetraen-1-ol | C₂₀H₃₄O | 290.49 | | |
| 1-Heneicosanol | C₂₁H₄₄O | 312.58 | 69.5 | |
| 1-Docosanol | C₂₂H₄₆O | 326.61 | 73.5 | 180/0.03 |
| (behenyl alcohol) | | | | |
| 1-3-cis-Docosen-1-ol | C₂₂H₄₄O | 324.59 | 34.5 | 241/1.3 |
| (erucyl alcohol) | | | | |
| 1-3-trans-Docosen-1-ol | C₂₂H₄₄O | 324.59 | 53.5 | 241/1.1 |
| (brassidyl alcohol) | | | | |

Penetration enhancers used with further preference are diesters and diethers of polyethylene glycol 6 to 12 with C₈-C₁₈ fatty alcohols and/or C₈-C₁₈ fatty acids.

By polyethylene glycols are meant polyalkylene glycols which belong to the class of the polyethers and are of general formula:

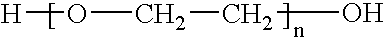

Polyethylene glycols are produced industrially by a basic catalyzed polyaddition reaction of ethylene oxide (oxirane) in systems containing usually small amounts of water, with ethylene glycol as starter molecule. They have molar masses in the range of from about 200 to 5 000 000 g/mol, corresponding to degrees of polymerization n of from about 5 to >100 000.

Enhancers used with further preference are propylene glycol diesters with C₈-C₁₈ fatty alcohols.

Enhancers used with further preference are glycerol diesters and triesters with C₈-C₁₈ fatty alcohols.

Suitability as matrix is possessed by absorbent, self-adhesive polyurethanes, in foamed or unfoamed form, which may further include fillers or auxiliaries, such as absorbent materials.

DE 196 18 825 A1 relates to suitable polyurethanes and discloses hydrophilic, self-adhesive polyurethane gels which consist of
a) polyetherpolyols having 2 to 6 hydroxyl groups and OH numbers of from 20 to 112 and an ethylene oxide (EO) content of >10% by weight,
b) antioxidants,
c) bismuth(III)carboxylates based on carboxylic acids having 2 to 18 C atoms and soluble in the polyols a), as catalysts, and
d) hexamethylene diisocyanate,
   with a product of the functionalities of the polyurethane-forming components a) and d) of at least 5.2, where the amount of catalyst c) is from 0.005 to 0.25% by weight based on the polyol a), the amount of antioxidants b) is in the range from 0.1 to 1.0% by weight based on polyol a), and a ratio of free NCO groups of component d) to free OH groups of component a) (isocyanate index) is chosen in the range from 0.30 to 0.70.

Polyetherpolyols preferably having 3 to 4, very particularly preferably 4, hydroxyl groups and having an OH number in the range from 20 to 112, preferably 30 to 56, are employed. The ethylene oxide content in the polyetherpolyols employed according to the invention is preferably ≧20% by weight.

The polyetherpolyols are known as such per se and are prepared by self-polymerization of epoxides such as ethylene oxide, propylene oxide, butylene oxide or tetrahydrofuran, or by addition of these epoxides, preferably of ethylene oxide and propylene oxide—where appropriate mixed together or separately and consecutively—onto starter components having at least two reactive hydrogen atoms, such as water, ethylene glycol, propylene glycol, diethylene glycol, dipropylene glycol, glycerol, trimethylolpropane, pentaerythritol, sorbitol or sucrose. Representatives of the high molecular weight polyhydroxy compounds mentioned for use are listed for example in High Polymers, Vol. XVI, “Polyurethanes, Chemistry and Technology” (Saunders-Frisch, Interscience Publishers, New York, Vol. 1, 1962, pages 32-42).

The isocyanate component employed is monomeric or trimerized hexamethylene diisocyanate, or hexamethylene diisocyanate which has been modified by biuret, uretdione, allophanate groups or by prepolymerization with polyetherpolyols or mixtures of polyetherpolyols based on known starter components having 2 or >2 reactive H atoms and epoxides such as ethylene oxide or propylene oxide of an OH number of ≦850, preferably 100 to 600. The use of modified hexamethylene diisocyanate is preferred, in particular hexamethylene diisocyanate modified by prepolymerization with polyetherdiols of OH number 200 to 600. It is very particularly preferred for the hexamethylene diisocyanate to be modified with polyetherdiols of OH number 200-600 whose residual content of monomeric hexamethylene diisocyanate is below 0.5% by weight.

Suitable catalysts for the polyurethane gels of the invention are bismuth(III)carboxylates which are based on linear, branched, saturated or unsaturated carboxylic acids having 2 to 18, preferably 6 to 18, C atoms and which are soluble in the anhydrous polyetherpolyols a). Bi(III) salts of branched saturated carboxylic acids having tertiary carboxyl groups, such as 2,2-dimethyloctanoic acid (for example Versatic acids, Shell), are preferred. Preparations of these Bi(III) salts in excess proportions of these carboxylic acids are very suitable. A solution of 1 mol of the Bi(III) salt of versatic 10 acid (2,2-dimethyloctanoic acid) in an excess of 3 mol of this acid with a Bi content of about 17% has proved outstandingly suitable.

The catalysts are preferably employed in amounts of from 0.03 to 0.1% by weight based on the polyol a).

Antioxidants suitable for the polyurethane gels of the invention are, in particular, sterically hindered phenolic stabilizers such as BHT (2,6-di-tert-butyl-4-methylphenol), Vulkanox BKF (2.2 min-methylenebis(6-tert-butyl-4-methylphenol) (Bayer AG), Irganox 1010 (pentaerythrityl tetrakis[3-(3,5-ditert-butyl-4-hydroxyphenyl)propionate]), Irganox 1076 (octadecyl 3-(3,5-ditert-butyl-4-hydroxyphenyl)propionate) (Ciba-Geigy) or tocopherol (vitamin E). Those of the α-tocopherol type are preferably employed.

The antioxidants are preferably employed in amounts of from 0.15 to 0.5% by weight based on the polyol a).

The isocyanate index (ratio of the free NCO groups employed in the reaction to the free OH groups) of the polyurethane gel compositions of the invention is, depending on the functionality of the isocyanate and polyol components employed, in the range from 0.30 to 0.70, preferably in the range from 0.45 to 0.60. The isocyanate index necessary for gel formation can be estimated very simply from the following formula:

$${f}_{( polyol )} \cdot ( {f}_{( isocyanate )} - 1 ) \cdot index \approx 2$$

$$index \approx \frac{2}{{f}_{( polyol )} \cdot ( {f}_{( isocyanate )} - 1 )}$$

f: functionality of the isocyanate or polyol component

The isocyanate index actually to be used may vary by up to +20% from the calculated value depending on the desired tack or elasticity of the gel.

The polyurethane gel compositions of the invention are prepared by conventional processes as described, for example, in Becker/Braun, Kunststoff-Handbuch, Vol. 7, Polyurethane, pages 121 et seq., Carl-Hauser, 1983.

Further polyurethanes which are preferably employed are those disclosed in EP 0 665 856 B1.

The hydrophilic polyurethane gel foams are obtainable according to this from
1. a polyurethane gel which comprises
(A) 25-62% by weight, preferably 30-60% by weight, particularly preferably 40-57% by weight, based on the total of (A) and (B), of a covalently crosslinked polyurethane as high molecular weight matrix and
(B) 75-38% by weight, preferably 70-40% by weight, particularly preferably 60-43% by weight, based on the total of (A) and (B), of one or more polyhydroxyl compounds which are firmly bound in the matrix by secondary valence forces and have an average molecular weight between 1000 and 12000, preferably between 1500 and 8000, particularly preferably between 2000 and 6000, and an average OH number between 20 and 112, preferably between 25 and 84, particularly preferably between 28 and 56, as liquid dispersant, the dispersant being essentially free of hydroxyl compounds with a molecular weight below 800, preferably below 1000, particularly preferably below 1500, and, where appropriate,
(C) 0-100% by weight, based on the total of (A) and (B), of fillers and/or additives,
   and which is obtainable by reacting a mixture of
a) one or more polyisocyanates,
b) one or more polyhydroxyl compounds with an average molecular weight between 1000 and 12000, and with an average OH number between 20 and 112,
c) where appropriate catalysts or accelerators for the reaction between isocyanate groups and hydroxyl groups and, where appropriate,
d) fillers and additives known per se from polyurethane chemistry,
this mixture being essentially free of hydroxyl compounds with a molecular weight below 800, the average functionality of the polyisocyanates (F_{I}) being between 2 and 4, the average functionality of the polyhydroxyl compound (F_{P}) being between 3 and 6, and the isocyanate index (K) being given by the formula

$$K = \frac{300 \pm X}{( {F}_{I} \cdot {F}_{P} ) - 1} + 7$$

in which X≦120, preferably X≦100, particularly preferably X≦90, and the index K has values between 15 and 70, where the stated averages of molecular weight and OH number are to be understood as number averages,
2. a water-absorbing material and
3. a non-aqueous foaming agent.

The polyurethane gels can be prepared from the starting compounds known in polyurethane chemistry by processes known per se, as described for example in DE 31 03 499 A1, DE 31 03 500 A1 and EP 0 147 588 A1. However, it is essential that the above-defined conditions are complied with in the selection of the gel-forming components because, otherwise, tack-free, elastic gels are obtained in place of self-adhesive gels.

Preferred polyhydroxy compounds are polyetherpolyols like those mentioned in detail in the abovementioned publications.

Both (cyclo)aliphatic and aromatic isocyanates are suitable as polyisocyanate components. Preferred (cyclo)aliphatic polyisocyanates are 1,6-hexamethylene diisocyanate and its biurets and trimers, and hydrogenated diphenylmethane diisocyanate (“MDI”) types. Preferred aromatic polyisocyanates are those obtained by distillation, such as MDI mixtures of 4,4′ and 2,4′ isomers or 4,4′-MDI, and tolylene diisocyanate (“TDI”) types.

The diisocyanates may be chosen in particular for example from the group of unmodified aromatic or aliphatic diisocyanates or else from modified products formed by prepolymerization with amines, polyols or polyetherpolyols.

The polyurethane composition may be unfoamed, foamed, unfilled or employed with additional fillers, such as, for example, superabsorbents, titanium dioxide, zinc oxide, plasticizers, dyes etc. It is additionally possible to use hydrogels in semisolid to solid form with active constituents for the central zone.

The polyurethane gels may, where appropriate, comprise additives known per se from polyurethane chemistry, such as, for example, inorganic- or organic-based fillers and short fibers, metal pigments, surface-active substances or liquid extenders such as substances having a boiling point of above 150° C.

Examples of organic fillers which may be mentioned are barytes, chalk, gypsum, kieserite, soda, titanium dioxide, cerium oxide, quartz sand, kaolin, carbon black and hollow microspheres.

Organic fillers which can be employed are, for example, powders based on polystyrene, polyvinyl chloride, urea-formaldehyde and polyhydrazodicarbonamide. Suitable short fibers are, for example, glass fibers with a length of 0.1-1 mm or fibers of organic origin, such as, for example, polyester or polyamide fibers. Metal powders such as, for example, iron or copper powder can likewise also be used in the gel formation. In order to confer the desired color on the gels, the organic- or inorganic-based dyes or color pigments known per se for the coloring of polyurethanes can be used, such as, for example, iron oxide or chromium oxide pigments, phthalocyanine- or monoazo-based pigments. Examples of surface-active substances which may be mentioned are cellulose powder, activated carbon and silica products.

The adhesive properties of the gels can be modified by adding where appropriate additions of polymeric vinyl compounds, polyacrylates and other copolymers customary in adhesives technology, or else adhesives based on natural substances up to a content of 10% by weight based on the weight of the gel composition.

Preferred water-absorbing materials are the water-absorbing salts, known as superabsorbents, of polyacrylates and copolymers thereof, especially the sodium or potassium salts. They may be uncrosslinked or crosslinked and are also available as commercial products. Particularly suitable products are those disclosed in DE 37 13 601 A1, as well as superabsorbents of the new generation having only small contents of water removable by drying and high swelling capacity under pressure.

Preferred products are slightly crosslinked polymers based on acrylic acid/sodium acrylate. Such sodium polyacrylates are obtainable as Favor T (Chemische Fabrik Stockhausen GmbH, Germany).

Further absorbents, for example carboxymethylcellulose and karaya, are likewise suitable.

The degree of foaming can be varied within wide limits by the incorporated amounts of foaming agent.

It is further preferred for the matrix to have a thickness of from 10 to 1 000 μm, very particularly 30 to 300 μm.

A large number of substance groups, which are free from hydroxyl, carboxyl or amine functionalities that are reactive in relation to the polyurethane crosslinking reaction, are used as active ingredients, for example essential oils, cosmetic skin-care additives, pharmaceutically active substances or antiseptics.

Transdermal therapeutic systems doped with essential oils and their constituents (for example eucalyptus oil, peppermint oil, camphor or menthol) have a long-term therapeutic effect on colds, headaches and other indications. Surprisingly, the hydroxyl functionality in menthol does not disrupt the polyurethane crosslinking reaction, a fact which may be explained by the lesser reactivity of the secondary OH group in the menthol molecule.

Concentrates obtained from plants and employed as natural raw materials mainly in the perfume industry and foodstuffs industry, and consisting more or less of volatile compounds such as, for example, true essential oils, citrus oils, absolutes, resinoids, are known as essential oils.

The term is often also used for the volatile constituents still present in the plants. However, essential oils in the real sense mean mixtures of volatile components produced by steam distillation from plant raw materials.

True essential oils consist exclusively of volatile components whose boiling points are mainly between 150 and 300° C. Unlike, for example, fatty oils, they therefore do not leave permanent transparent greasy spots behind when dabbed onto filter paper. Essential oils comprise mainly hydrocarbons or monofunctional compounds such as aldehydes, esters, ethers and ketones.

Parent compounds are mono- and sesquiterpenes, phenylpropane derivatives and longer-chain aliphatic compounds.

In some essential oils, one constituent predominates (for example eugenol comprising more than 85% of clove oil), while others have extremely complex compositions. The organoleptic properties are often determined not by the main components but by subsidiary or trace constituents, such as, for example, by the 1,3,5-undecatrienes and pyrazines in galbanum oil. The number of identified components in many of the commercially significant essential oils is up in the hundreds. Very many constituents are chiral, with one enantiomer very often predominating or being exclusively present, such as, for example, (−)-menthol in peppermint oil or (−)-linalyl acetate in lavender oil.

In an advantageous embodiment, the matrix comprises from 0.1 to 20% by weight, in particular 1 to 10% by weight, of essential oils which are chosen in particular from the group of eucalyptus oil, peppermint oil, camomile oil, camphor, menthol, citrus oil, cinnamon oil, thyme oil, lavender oil, clove oil, teatree oil, cajeput oil, niaouli oil, kanuka oil, manuka oil, dwarf pine oil.

Citrus oils are essential oils obtained from the peel of citrus fruits (bergamot, grapefruit, lime, mandarin, orange, lemon), often also called agrumen oils.

Citrus oils consist largely of monoterpene hydrocarbons, mainly limonene (exception: bergamot oil, which contains only about 40%).

Camphor means 2-bornanone, 1,7,7-trimethylbicyclo[2.2.1]heptan-2-one, see figure below.

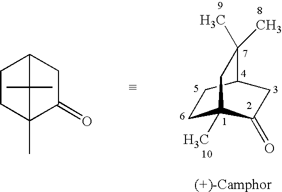

Peppermint oils are essential oils obtained by steam distillation from the leaves and flowers of various types of peppermint, occasionally also those from Mentha arvensis.

Menthol has three asymmetric C atoms and accordingly exists in four diastereomeric pairs of enantiomers (cf. the formulae; the other four enantiomers are the corresponding mirror images).

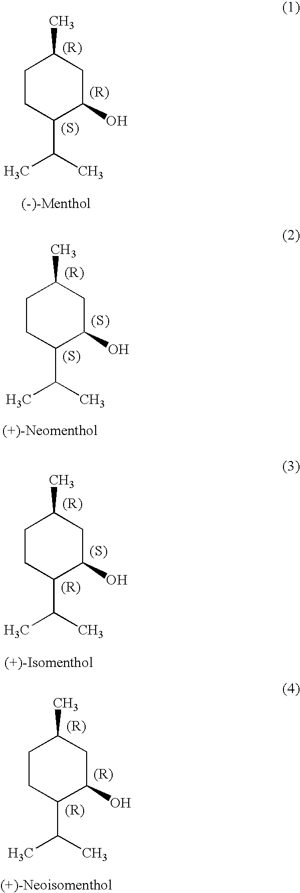

The diastereomers, which can be separated by distillation, are referred to as neoisomenthol, isomenthol, neomenthol [(+) form: constituent of Japanese peppermint oil) and menthol. The most important isomer is (−)-menthol (levomenthol), shining prisms with a strong peppermint-like odor.

When menthol is rubbed into the skin (especially on the forehead and temples), it causes a pleasant cool sensation as a result of surface anesthesia and stimulation of the cold-sensitive nerves in migraine and the like; in fact, the relevant areas show a normal or elevated temperature. These effects are not possessed by the other menthol isomers.

It is furthermore possible and advantageous to add cosmetic skin-care additives to the matrix, in particular to the extent of 0.2 to 10% by weight, very especially 0.5 to 5% by weight.

The cosmetic skin-care additives (one or more compounds) can very advantageously be chosen according to the invention from the group of lipophilic additives, especially from the following group:
azulene, vitamins, vitamin A palmitate, caffeine.

It is also advantageous to choose the additives from the group of superfatting substances, for example Purcellin oil, Eucerit® and Neocerit®.

The additive(s) are furthermore particularly advantageously chosen from the group of NO synthase inhibitors, especially if the preparations of the invention are to be used for the treatment and prophylaxis of the symptoms of intrinsic and/or extrinsic skin aging, and for the treatment and prophylaxis of the harmful effects of ultraviolet radiation on the skin.

The preferred NO synthase inhibitor is nitroarginine.

It is also advantageous to choose the additive(s) from the group of ubiquinones and plastoquinones.

Ubiquinones are distinguished by the structural formula 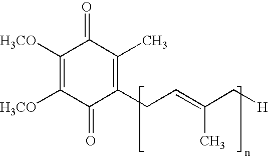
and represent the most widespread and thus best-investigated bioquinones. Ubiquinones are referred to as Q-1, Q-2, Q-3 etc. depending on the number of isoprene units linked in the side chain, or as U-5, U-10, U-15 etc. according to the number of C atoms. They occur preferentially with particular chain lengths, for example with n=6 in some microorganisms and yeasts. Q10 predominates in most mammals, including humans.

Coenzyme Q10 is particularly advantageous, and is characterized by the following structural formula:

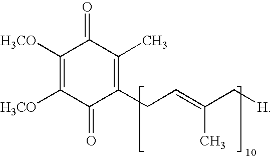

Plastoquinones have the general structural formula

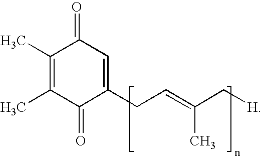

Plastoquinones differ in the number n of isoprene residues and are designated correspondingly, for example PQ-9 (n=9). Other plastoquinones with different substituents on the quinone ring also exist.

Creatine and/or creatine derivatives are also preferred additives for the purposes of the present invention. Creatine is distinguished by the following structure:

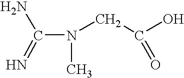

Preferred derivatives are creatine phosphate and creatine sulfate, creatine acetate, creatine ascorbate and the derivatives esterified with mono- or polyfunctional alcohols on the carboxyl group.

The list of additives and additive combinations mentioned and being usable in the preparations of the invention is, of course, not intended to be limiting, save for the criterion of the isocyanate-reactive hydroxyl or carboxyl group. The additives can be used single or in any combinations with one another.

It is then possible to add pharmaceutically active substances to the matrix of the active ingredient-containing matrix patch, at preferably up to 40% by weight, in particular 0.01 to 25% by weight, very particularly 0.1 to 10% by weight.

Typical active ingredients are—without claiming completeness within the scope of the present invention:

| Indication: | Active substance |
|---|---|
| Antimycotics | Natifine |
| | ((E)-N-Cinnamyl-N-methyl-1- naphthalenemethanamine) |
| | 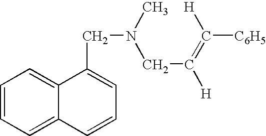 |
| | Amorrolfine |
| ((±)-cis-2,6-Dimethyl-4-[2-methyl-3- (4-tert-pentylphenyl)-propyi]morpholine) | |
| | Tolnaftate |
| | (O-(2-Naphthyl)-N-methyl- N-m-tolyl-thiocarbamate) |
| | 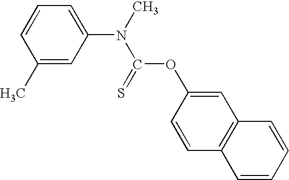 |
| | Clotrimazole |
| | (1 -[(2-Chlorophenyl)diphenylmethyl]-1H-imidazole) |
| | 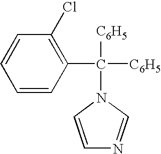 |
| Antiseptics | Triclosan |
| | Ethacridine |
| | Chlorhexidine |
| | Hexetidine |
| | Dodicin |
| | Iodine |
| Non-steroidal anti- | Methyl salicylate |
| inflammatory drugs | Etofenamate |
| | Indomethacin |
| | ([1-(4-Chlorobenzoyl)-5-methoxy-2 -methyl-1H-indol-3-yl]acetic acid |
| | 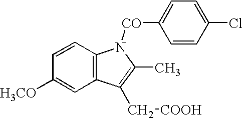 |
| Antipruritics | Crotamiton |
| Local anesthetics | Benzocaine |
| Antipsoriatics | |
| Keratolytics | Urea |

Further active ingredients which promote wound healing, such as silver sulfadiazine, can likewise be employed.

With particular advantage and in the sense of the invention it is possible to mention also hyperemic active ingredients such as natural active ingredients from cayenne pepper or synthetic active ingredients such as nonivamide, nicotinic acid derivatives, preferably benzyl nicotinate or propyl nicotinate, and anti-inflammatory agents and/or analgesics.

Examples which may be mentioned are capsaicin

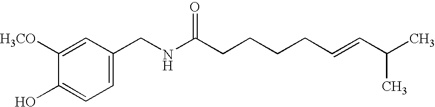

### [8-Methyl-trans-6-nonenoic Acid 4-hydroxy-3-methoxybenzyl Amide]

Nonivamide 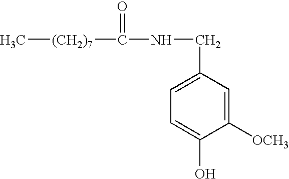
Nicotinic Acid Benzyl Ester

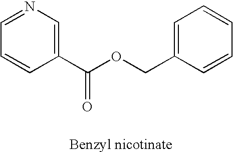

Active ingredients which should be emphasized as particularly important are the disinfectants and antiseptics, so that use thereof in the matrix is to be stressed once again.

Substances designated as disinfectants are those suitable for disinfection, i.e. for controlling pathogenic microorganisms (for example bacteria, viruses, spores, microfungi and molds), in particular generally by use on the surface of the skin, clothing, equipment, rooms, but also drinking water, foodstuffs, seeds (dressing) and as soil disinfectant.

Disinfectants particularly for local use, for example for disinfecting wounds, are also referred to as antiseptics.

As antiseptic use may be made in particular of the derivatives of lactic acid, such as esters and also oligolactic and polylactic acid.

By lactic esters are meant the esters frequently called lactates of the respective alcohol component, having the general formula 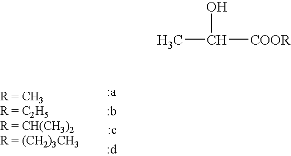
the majority of which are low-melting products or are liquid at 20° C., and which, except for the lower alkyl esters, are sparingly soluble in water but readily soluble in alcohol and ethers.

The following lactic esters are differentiated:
(a) lactic acid methyl ester (methyl lactate), C4H8O3, Mᵣ 104.10, boiling point 145° C.
(b) lactic acid ethyl ester (ethyl lactate), C5H10O3, Mᵣ 118.13, D. 1.03, boiling point 154° C.
(c) lactic acid isopropyl ester (isopropyl lactate), C6H12O3, Mᵣ 132.15, D. 0.9980, boiling point 167° C.
(d) lactic acid butyl ester (butyl lactate), C7H14O3, Mᵣ 146.18, D. 0.9803, boiling point 187° C.

Polylactic acid (polylactide) is a polyester based on lactic acid, from whose lactide it can be prepared by means of ring-opening polymerization.

The matrix then, in a further advantageous embodiment, comprises an in particular hydrophilic filler based on cellulose and derivatives thereof, the average particle size of which is in the range from 20 to 60 μm, since it has surprisingly been found in the selection of the fillers that, in particular, fillers based on silicon dioxide or cellulose are suitable, the latter having an isotropic morphology and not being prone to swelling on contact with water. Fillers particularly suitable in this connection are those having a particle size of less than or equal to 100 μm.

The use of hydrophilic fillers in a nonpolar matrix is known in the literature. They are described in EP 0 186 019 A1 explicitly for use in transdermal therapeutic systems. However, in this case only to a concentration of 3 to 30% by weight, without details of these fillers being mentioned. Experience shows that systems with a filler content of more than 30% by weight show a marked loss of tack and become hard and brittle. They thereby lose the fundamental requirement of a transdermal therapeutic system.

Fillers based on microcrystalline or amorphous cellulose are preferably employed in considerably higher concentrations without adversely affecting the adhesive properties, especially if they have an isotropic morphology with a particle size not exceeding 100 μm. Higher filler contents are desirable to improve the wearing properties, especially on long-lasting and repeated use.

The matrix may additionally have on the side facing away from the skin or wound a covering of a backing material, for example consisting of sheets (for example of PU, polyester, PE or PP), nonwovens, wovens, foams, metallized sheets, composite materials, cotton etc.

Preference from the group of suitable backing materials is given to the occlusive sheets.

A metallocene polyethylene nonwoven is also suitable, for example.

The metallocene polyethylene nonwoven preferably has the following properties:
a basis weight of from 40 to 200 g/m², in particular from 60 to 120 g/m², and/or
a thickness of from 0.1 to 0.6 mm, in particular from 0.2 to 0.5 and/or
a lengthwise ultimate tensile stress elongation of from 400 to 700% and/or
a transverse ultimate tensile stress elongation of from 250 to 550%.

It is thus possible to employ as backing materials known webs which are mechanically consolidated, either by overstitching with separate yarns or by interlooping.

In the first case, the resulting structures are web-yarn stitchbonds. They are produced from a fiber web, which may be, for example, of cross-plated configuration, by overstitching with separate yarns in pillar-stitch formation or tricot formation.

These webs are known under the name “Maliwatt” (from Malimo) or Arachne.

The second type of consolidation likewise preferably starts from a cross-plated web.

During the consolidation operation, needles draw out fibers from the web itself and form them into loops, with stitches being formed in pillar-stitch formation. This web stitchbond is widely used under the name “Maliviies”, likewise from Malimo.

A review of the various types of mechanically consolidated fiber nonwovens can be found in the article “Kaschierung von Autopolsterstoffen mit Faserviiesen” by G. Schmidt, Melliand Textilberichte June/1992, pages 479 to 486.

It can be stated in summary that suitable backing materials are all rigid and elastic sheet-like structures made from synthetic and natural raw materials. Preferred backing materials can be employed in such a way that they comply with the properties of a functionally appropriate dressing. Textiles mentioned by way of example are those such as wovens, knits, lays, nonwovens, laminates, nets, sheets, foams and papers. In addition, these materials may be pretreated and/or aftertreated. Common pretreatments are corona and hydrophobization; customary aftertreatments are calendering, thermal conditioning, laminating, punching and sheathing.

In one preferred embodiment the matrix has been applied to a backing material, preferably in such a way that the periphery of the backing material at least in part is not covered by the matrix.

A further possibility is for a layer of adhesive composition, in particular based on PU, acrylates or rubber, to be present between the matrix and the backing material.

Finally, the matrix and/or the backing material coated with the adhesive composition may, if the matrix is not present on the whole area of the backing material, be covered with the usual release paper.

The matrix patch of the invention may have any desired shape, with preference for a regular shape such as rectangular, square, circular or oval.

Preferred embodiments of the subject matter of the invention, and several figures, are described by way of example below, without thereby wishing unnecessarily to restrict the invention.

### EXAMPLES 1 TO 7

### Production of Active Ingredient-Containing Polyurethanes

The active ingredient nonivamide (NVA, Boehringer Ingelheim) is melted in a heat cabinet and mixed homogeneously with isopropyl palmitate (IPP, Pronova Oleochemicals), with stirring.

The Levagel (polyetherpolyol from Bayer, Leverkusen) and the Desmodur (hexamethylene diisocyanate-based polyisocyanate from Bayer, Leverkusen) are weighed out into a vessel and mixed homogeneously with the nonivamide/isopropyl palmitate mixture for the duration of a few minutes, with stirring.

Following the addition of Coscat 83 (bismuth salt from C. H. Erbslöh) the mixture is stirred for homogeneity for a minute and then the still liquid mass is applied by means of a coating bar with a defined slot width between a backing sheet (polyurethane film, Beiersdorf, Hamburg or polyethylene film, BP Chemicals) and a silicone paper (or silicone sheet).

Quantities of the formulations for examples 1 to 7:

| Ex- | | | | | Coscat | Application | |
|---|---|---|---|---|---|---|---|
| am- | NVA | IPP | Levagel | Desmodur | 83 | rate | Back- |
| ple | g | g | g | g | g | g/m² | ing |
| 1 | 8.5 | 214.6 | 707.2 | 64.4 | 5.3 | 190 | PE |
| 2 | 15.1 | 203.7 | 711.0 | 64.7 | 5.5 | 96 | PE |
| 3 | 20.0 | 250.3 | 664.2 | 60.5 | 5.0 | 95 | PE |
| 4 | 20.0 | 250.3 | 664.4 | 60.3 | 5.0 | 281 | PE |
| 5 | 40.0 | 250.3 | 646.0 | 58.8 | 4.9 | 329 | PE |
| 6 | 18.3 | 0 | 894.3 | 81.4 | 6.0 | 500 | PE |
| 7 | 15.5 | 208.9 | 706.0 | 64.3 | 5.3 | 96 | PU |

### EXAMPLES 1 TO 7

### Active Ingredient Release

For investigating the release characteristics, specimens on pig's skin are prepared and the release is determined quantitatively after 24 hours.

| | Epidermis | Dermis | Receptor phase | Total release to skin |
|---|---|---|---|---|
| Example | μg/cm² | μg/cm² | μg/cm² | μg/cm² |
| 1 | 0.08 | 0.07 | 0 | 0.15 |
| 2 | 0.49 | 0.46 | 0.05 | 1.00 |
| 3 | 0.62 | 0.98 | 0.05 | 1.65 |
| 4 | 0.58 | 1.03 | 0 | 1.61 |
| 5 | 1.20 | 1.98 | 0.09 | 3.27 |
| 6 | 0.24 | 0.17 | 0.04 | 0.45 |
| 7 | 0.18 | 0.04 | 0 | 0.22 |

### Example 2

### Release of NVA to Pig's Skin as a Function of Time

| Time of | | | |
|---|---|---|---|
| testing | Epidermis | Dermis | Σ Epidermis/Dermis |
| [h] | [μg/cm²] | [μg/cm²] | [μg/cm²] |
| 2 | 0.08 | 0.02 | 0.10 |
| 4 | 0.13 | 0.05 | 0.18 |
| 6 | 0.22 | 0.06 | 0.28 |
| 8 | 0.27 | 0.08 | 0.36 |
| 24 | 0.49 | 0.46 | 0.95 |

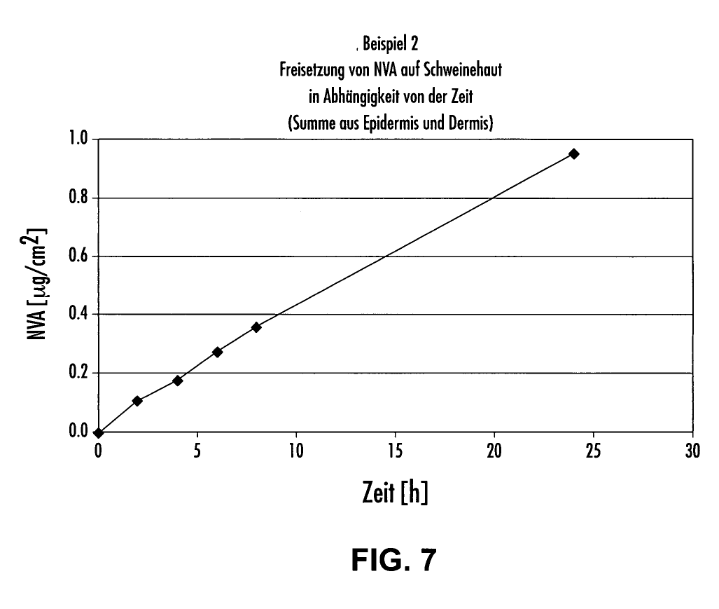

FIG. 7 shows the results from the above table in the form of a graph.

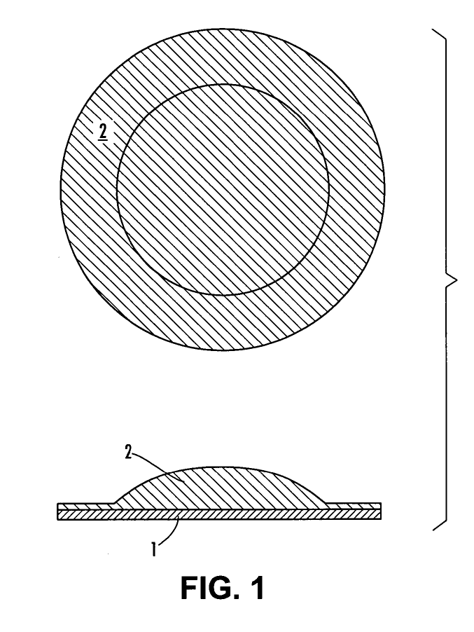

FIG. 1 illustrates a preferred geometric shape of the matrix patch.

The patch has a circular shape (diameter 100 mm), consists of a polyurethane matrix 2 which slopes down toward the edge. The polyurethane matrix 2 initially slopes down uniformly and terminates in a 20 mm-wide ring where the thickness remains constant. The polyurethane matrix 2 has a substantially semiconvex shape in the middle and is accordingly comparable to a semiconvex lens.

The thickness of the polyurethane matrix 2 is 2.3 mm in the middle and 0.7 mm at the edge.

Finally, the matrix 2 is covered by a siliconized paper 1 in order to prevent soiling or contamination of the matrix 2.

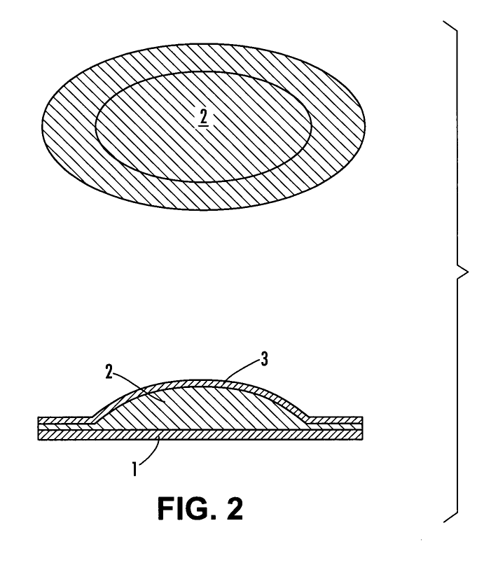

FIG. 2 illustrates a further preferred geometric shape of the matrix patch.

The patch has an ellipsoidal shape (length of the axes 42 mm and 68 mm respectively), consists of a polyurethane matrix 2 which slopes down toward the edge. The polyurethane matrix 2 initially slopes down uniformly and terminates in a ring approximately 11 mm wide where the thickness remains constant. The polyurethane matrix 2 has an essentially semiconvex shape in the middle and is accordingly comparable to a semiconvex lens.

The PU matrix 2 is covered on the side facing away from the skin with a PE sheet 3.

The thickness of the polyurethane matrix 2 together with PE sheet 3 is 1.6 mm in the middle and 0.3 mm at the edge.

Finally, the polyurethane matrix 2 is covered with a siliconized paper 1 in order to prevent soiling or contamination of the matrix 2.

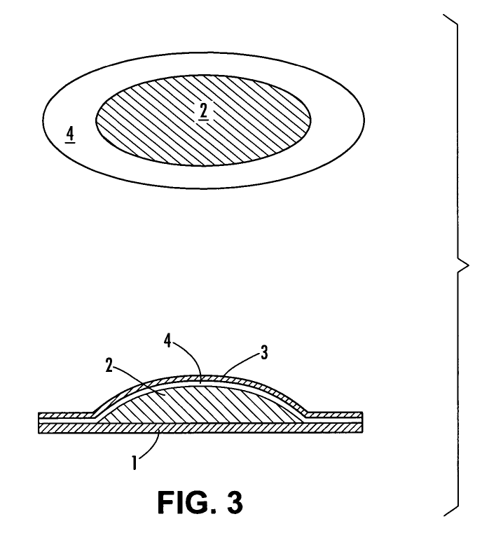

FIG. 3 illustrates a further preferred geometric shape of the matrix patch.

The patch has an ellipsoidal shape (length of the axes 110 mm and 65 mm respectively), consists of a polyurethane matrix 2 which slopes down toward the edge. The polyurethane matrix 2 has an essentially semiconvex shape and is accordingly comparable to a semiconvex lens with a length of the axes of 72 mm and 34 mm respectively.

The PU matrix 2 is covered on the side facing away from the skin with a PE sheet 3 which is coated over its whole area with the IPP-containing polyurethane-based adhesive layer 4. In the embodiment of the patch shown here, the entire periphery of the adhesive layer 4 is not covered by the polyurethane matrix 2. This results in two concentric zones of chemically different adhesive compositions 2, 4 which differ in terms of adherence, absorptivity and cushioning properties.

The thickness of the polyurethane matrix 2 together with PU sheet 3 and adhesive layer 4 is 1.3 mm in the middle and 0.15 mm at the edge.

Finally, the polyurethane matrix 2 is covered with a siliconized paper 1 in order to prevent soiling or contamination of the matrix 2.

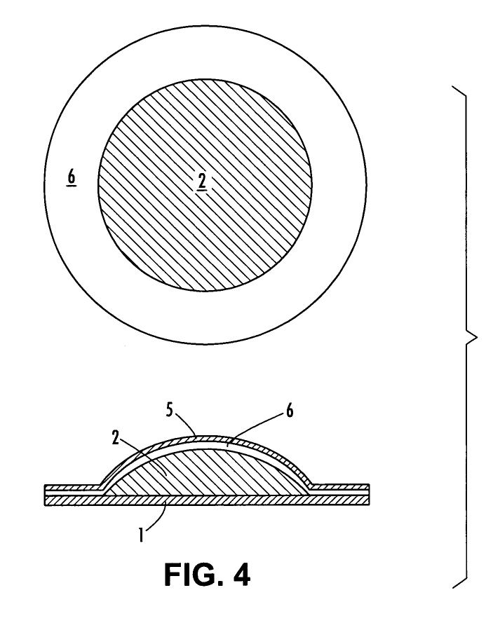

FIG. 4 illustrates a further preferred geometric shape of the matrix patch.

The patch has a circular shape (diameter 100 mm), consists of a foamed polyurethane matrix 2 which slopes down toward the edge. The polyurethane matrix 2 has an essentially semiconvex shape and is accordingly comparable to a semiconvex lens with a diameter of 60 mm.

The PU matrix 2 is covered on the side facing away from the skin with a PU sheet 3 which is coated over its whole area with an acrylate-based adhesive layer 6. In the embodiment of the patch shown here, the entire periphery of the adhesive layer 6 is not covered by the polyurethane matrix 2. This results in two concentric zones of chemically different adhesive compositions 2, 6, which differ in terms of adherence, absorptivity and cushioning properties.

The thickness of the polyurethane matrix 2 together with PU sheet 3 and adhesive layer 6 is 1.5 mm in the middle and 0.1 mm at the edge.

Finally, the polyurethane matrix 2 is covered with a siliconized paper 1 in order to prevent soiling or contamination of the matrix 2.

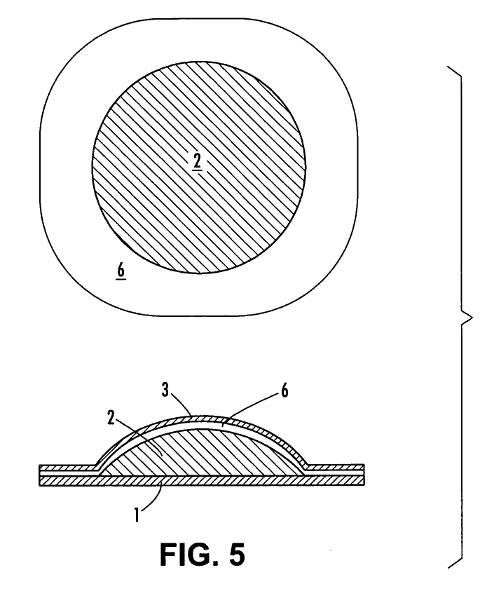

FIG. 5 illustrates a further preferred geometric shape of the wound dressing.

The patch has a square shape, with the corners of the square being rounded off (diameter of the square 50 mm), consists of a water vapor-permeable foamed polyurethane matrix 2 which slopes down toward the edge. The polyurethane matrix 2 has an essentially semiconvex shape and is circular, and is accordingly comparable to a semiconvex lens with a diameter of 33 mm.

The PU matrix 2 is covered on the side facing away from the skin with a PU sheet 3 which is coated over its whole area with a rubber-based adhesive layer 6. In the embodiment of the patch shown here, the entire periphery of the adhesive layer 6 is not covered by the polyurethane matrix 2. This results in two concentric zones of chemically different adhesive compositions 2, 6, which differ in terms of adherence, absorptivity and cushioning properties.

The thickness of the polyurethane matrix 2 together with PU sheet 3 and adhesive layer 6 is 1.5 mm in the middle and 0.1 mm at the edge.

Finally, the polyurethane matrix 2 is covered with a siliconized paper in order to prevent soiling or contamination of the matrix 2.

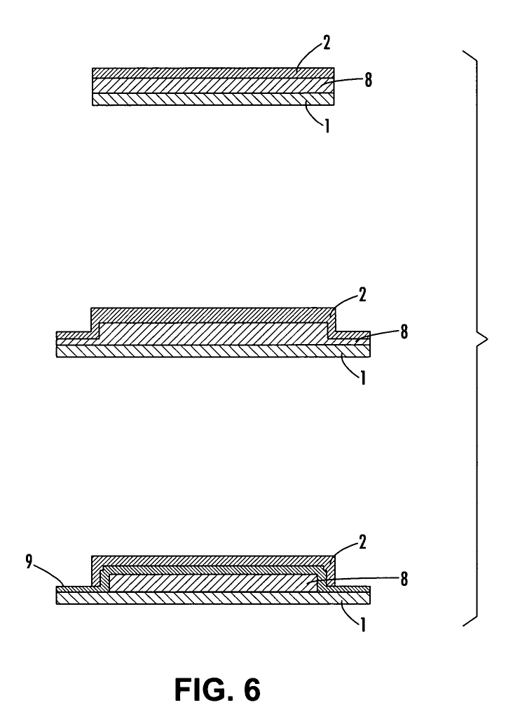

FIG. 6 shows three further embodiments of a matrix patch of the invention, specifically in cross section.

In the first embodiment of the three, the matrix patch consists of three individual layers. The doped wound pad made of polyurethane 2, the matrix 2, is covered over its whole area with a backing material 8 on the side facing away from the wound or skin. Polymer sheets, nonwovens, wovens and combinations thereof, and sheets or textile materials made of polymers such as polyethylene, polypropylene, polyurethane or else natural fibers, are used as backing material 8.

On the side facing the wound or skin, the self-adhesive matrix 2 is covered over its whole area with a release paper 1.

In the second embodiment of the matrix patch, the matrix 2 has in the center of the patch a relatively large layer thickness, while they are thinly shaped in the edge region of the patch.

In the third embodiment, an additional adhesive coating 9 applied to the whole area of the backing material 8 is present between the matrix 2 and the backing material 8.

Unlike the matrix patches in the first and second embodiment, in this case the matrix 2 does not extend over the entire area of the backing material 8. No matrix 2 is applied in the edge region of the backing material 8.

FIG. 7 documents the release of NVA to pig's skin.

In order to investigate the release characteristics, samples on pig's skin are prepared and the release is determined quantitatively after 2, 4, 6, 8 and 24 hours. The graph shows the quantity of NVA released in total to epidermis and dermis. A uniform, linear increase over the 24-hour period is evident. From this it is clear that the active ingredient is delivered in a controlled, sustained manner into the skin to develop its activity there.

1. A self-adhesive matrix patch for controlled delivery of active ingredients to the skin comprising a polyurethane gel matrix, wherein the polyurethane gel matrix comprises at least one active ingredient and at least one penetration enhancer.

2. The matrix patch as claimed in claim 1, wherein the total concentration of the at least one penetration enhancer is up to 30% by weight, based on the total weight of the polyurethane gel matrix.

3. The matrix patch as claimed in claim 1, wherein the at least one penetration enhancers includes at least one lipophilic solubilizer or enhancer.

4. The matrix patch as claimed in claim 1, wherein the polyurethane gel matrix is in foamed form.

5. The matrix patch as claimed in claim 1, wherein the at least one active ingredients includes at least one ingredient selected from the group consisting of essential oils, cosmetic skin-care additives, pharmaceutically active substances and antiseptics.

6. The matrix patch as claimed in claim 1, wherein the total concentration of the at least one active ingredient is from 0.1 to 20% by weight, based on the total weight of the matrix.

7. The matrix patch as claimed in claim 1, wherein the thickness of the polyurethane gel matrix is from 10 to 2000 μm.

8. The matrix patch as claimed in claim 1, further comprising a backing material the adjacent the polyurethane gel matrix.

9. The matrix patch as claimed in claim 8, further comprising an adhesive layer between the polyurethane gel matrix and the backing material.

10. The matrix patch as claimed in claim 8, wherein the backing material comprises an occlusive sheet.

11. The matrix patch as claimed in claim 10, wherein the backing material is selected from the group consisting of polyethylene, polypropylene, and polyester.

12. The matrix patch as claimed in claim 2, wherein the total concentration of the at least one penetration enhancer is from 5 to 15% by weight, based on the total weight of the polyurethane gel matrix.

13. The matrix patch as claimed in claim 1, wherein the at least one penetration enhancer includes at least one fatty acid ester.

14. The matrix patch as claimed in claim 13, wherein the at least one fatty acid ester is selected from the group consisting of decyl oleate, isopropyl myristate, isopropyl palmitate, and 2-octyldodecanol.

15. The matrix patch as claimed in claim 1, wherein the at least one penetration enhancer includes at least one fatty alcohol.

16. The matrix patch as claimed in claim 1, wherein the at least one penetration enhancer is selected from the group consisting of C₈-C₁₈ fatty acid esters of short-chain alcohols or fatty alcohols; diesters and diethers of polyethylene glycol 6 to 12 and C₈-C₁₈ fatty alcohols or C₈-C₁₈ fatty acids; propylene glycol diesters of C₈-C₁₈ fatty alcohols, and glycerol diesters or triesters of C₈-C₈ fatty alcohols.

17. The matrix patch as claimed in claim 1, wherein the at least one active ingredient includes at least one hyperemic active ingredient.

18. The matrix patch as claimed in claim 17, wherein the at least one hyperemic active ingredient includes at least one ingredient selected from the group consisting of nonivamide, benzyl nicotinate, propyl nicotinate, capsaicin, and cayenne pepper.

19. The matrix patch as claimed in claim 6, wherein the total concentration of the at least one active ingredient is from 2 to 10% by weight, based on the total weight of the matrix.

20. The matrix patch as claimed in claim 7, wherein the thickness of the polyurethane gel matrix is from 100 to 1,500 μm.

21. The matrix patch as claimed in claim 7, wherein the thickness of the polyurethane gel matrix is from 10 to 1,000 μm.

22. The matrix patch as claimed in claim 7, wherein the thickness of the polyurethane gel matrix is from 30 to 300 μm.

23. The matrix patch as claimed in claim 8, wherein said backing material does not cover at least a portion of the periphery of the polyurethane gel matrix.

24. The matrix patch as claimed in claim 1, wherein the at least one active ingredient includes nonivamide.

25. The matrix patch as claimed in claim 24, wherein the at least one penetration enhancer includes isopropyl palmitate.

26. A polyurethane gel matrix comprising at least one active ingredient and at least one penetration enhancer.

27. The gel matrix-as claimed in claim 26, wherein the total concentration of the at least one penetration enhancer is up to 30% by weight, based on the total weight of the polyurethane gel matrix.

28. The gel matrix-as claimed in claim 27, wherein the total concentration of the at least one penetration enhancer is from 5 to 15% by weight, based on the total weight of the polyurethane gel matrix.

28. The gel matrix-as claimed in claim 26, wherein the at least one penetration enhancer includes at least one lipophilic solubilizer or enhancer.

29. The gel matrix-as claimed in claim 26, wherein the at least one penetration enhancer includes at least one fatty acid ester.

30. The gel matrix-as claimed in claim 29, wherein the at least one fatty acid ester is selected from the group consisting of decyl oleate, isopropyl myristate, isopropyl palmitate, and 2-octyldodecanol.

31. The gel matrix as claimed in claim 26, wherein the at least one penetration enhancer includes at least one fatty alcohol.

32. The gel matrix as claimed in claim 26, wherein the at least one penetration enhancer is selected from the group consisting of C₈-C₁₈ fatty acid esters of short-chain alcohols or fatty alcohols; diesters and diethers of polyethylene glycol 6 to 12 and C₈-C₁₈ fatty alcohols or C₈-C₁₈ fatty acids; propylene glycol diesters of C₈-C₁₈ fatty alcohols, and glycerol diesters or triesters of C₈-C₁₈ fatty alcohols.

33. The gel matrix-as claimed in claim 26, wherein the polyurethane gel matrix is in foamed form.

34. The gel matrix-as claimed in claim 26, wherein the at least one active ingredient includes at least one ingredient selected from the group consisting of essential oils, cosmetic skin-care additives, pharmaceutically active substances, and antiseptics.

35. The gel matrix-as claimed in claim 26, wherein the at least one active ingredient includes at least one hyperemic active ingredient.

36. The gel matrix-as claimed in claim 35, wherein the at least one hyperemic active ingredient includes at least one ingredient selected from the group consisting of nonivamide, benzyl nicotinate, propyl nicotinate, capsaicin, and cayenne pepper.

37. The gel matrix-as claimed in claim 26, wherein the total concentration of the at least one active ingredient is from 0.1 to 20% by weight, based on the total weight of the matrix.

38. The gel matrix-as claimed in claim 26, wherein the total concentration of the at least one active ingredient is from 2 to 10% by weight, based on the total weight of the matrix.

39. The gel matrix-as claimed in claim 26, wherein the at least one active ingredient includes nonivamide.

40. The gel matrix-as claimed in claim 39, wherein the at least one penetration enhancer includes isopropyl palmitate.
